(19) United States
(12) Patent Application Publication
(10) Pub. No.: US 2019/0150741 A1
(43) Pub. Date: May 23, 2019

(54) EQUIPMENT AND METHODOLOGIES FOR INTRA-TUMORAL INJECTION

(71) Applicant: Weinberg Medical Physics, Inc., North Bethesda, MD (US)

(72) Inventor: Irving N. WEINBERG, North Bethesda, MD (US)

(73) Assignee: Weinberg Medical Physics, Inc.

(21) Appl. No.: 16/197,741

(22) Filed: Nov. 21, 2018

Related U.S. Application Data
(60) Provisional application No. 62/589,233, filed on Nov. 21, 2017.

(51) Int. Cl. A61B 5/00; A61B 5/055; A61N 2/00; A61K 41/00; A61K 47/69; A61K 35/768

(52) U.S. Cl. CPC A61B 5/0036; A61B 5/055; A61K 35/768; A61K 41/00; A61K 47/6941; A61N 2/002

(57) ABSTRACT

Disclosed embodiments provide a tool and methodologies for delivering therapeutic payload via a magnetic carrier to one or more target/tumor sites within a subject's body.

### CROSS REFERENCE AND PRIORITY CLAIM

This patent application claims priority to U.S. Provisional Application Provisional Patent Application No. Patent application Ser. No. 62/589,233, entitled “INTRA-TUMORAL INJECTION,” filed Nov. 21, 2017, the disclosure of which being incorporated herein by reference in its entirety.

### FIELD

Disclosed embodiments relate to the treatment of cancer and other diseases.

### BACKGROUND

Systemic administration of cancer therapeutics has been used in treating metastatic disease. Recently, intra-tumoral injection has been shown to be more successful than systemic therapy (see for example: C J Breitbach, B D Lichty, J C Bell. Oncolytic Viruses: Therapeutics with an Identity Crisis. EBioMedicine 9 (2016):31-36).

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to the more detailed description below.

Although the reason for the technical and medical advantage of intra-tumoral injection is not clear, potentially, intra-tumoral injection may assist the body in mounting an immune response to tumor cells that have dispersed elsewhere in the body from the injection site.

Accordingly, disclosed embodiments provide a tool and methodologies for intra-tumoral injection to enable delivery of drugs or other payload for medical treatment of a tumor within a subject's body.

Disclosed embodiments provide a non-invasive method and associated equipment for delivery of drugs or other payload for medical treatment to one or more tumors in a subject's body.

In accordance with disclosed embodiments, one or more magnetizable particles may be introduced non-invasively into one or more tumors within body structures in a subject.

In accordance with disclosed embodiments, at least one image-guidance component located in proximity to the one or more body structures may be used to direct, transport, concentrate and/or focus the one or more particles within the one or more body structures within a subject.

### BRIEF DESCRIPTION OF FIGURES

Further advantages, features and possibilities of using the present disclosed embodiments emerge from the description below in conjunction with the figures.

FIG. 1 illustrates one example of a disclosed embodiment where a part of a subject's body is imaged to enable image-guided delivery of one or more therapeutic drugs or other medical treatment payload.

FIG. 2 illustrates method operations performed in accordance with a disclosed embodiment, wherein a carrier with magnetic components and a payload are delivered with the aid of magnetic assemblies to a target location or region within a part of a subject's body.

FIG. 3 illustrates method operations performed in accordance with a disclosed embodiment, wherein walls of the carrier are dissolved and a payload is delivered to a target location or region within a part of a subject's body.

### DETAILED DESCRIPTION

The description of specific embodiments is not intended to be limiting of the present invention. To the contrary, those skilled in the art should appreciate that there are numerous variations and equivalents that may be employed without departing from the scope of the present invention. Those equivalents and variations are intended to be encompassed by the present invention.

In the following description of various invention embodiments, reference is made to the accompanying drawings, which form a part hereof, and in which is shown, by way of illustration, various embodiments in which the invention may be practiced. It is to be understood that other embodiments may be utilized and structural and functional modifications may be made without departing from the scope and spirit of the present invention.

For the purpose of the disclosed embodiments, the term “imaging,” includes imaging technology that utilize components to form an image using magnetic resonance or magnetic particle imaging. It should be understood that such components include coils or magnets (or electro-permanent magnets) that polarize protons or other nuclei or electrons in one or more structures to be imaged, wherein gradient and/or radiofrequency coils form an image. Thus, although not shown in detail herein, it should be understood that the disclosed embodiments may be used in conjunction with a support structure that may hold an imaging system and may contain other components needed to operate or move the imaging system, for example, wheels and/or batteries. Moreover, it should be understood that an associated display system is not shown but should be understood to be present in order to view images produced by the imaging system.

The terms “magnetic resonance imaging” and “magnetic particle imaging” are included under the overall term “magnetic imaging”. The term “magnetic imaging guidance” is understood to mean the sequence of imaging a target and a magnetic carrier in succession so as to guide propulsion of the magnetic carrier toward and/or into the target, for example in a closed-loop feedback situation.

It should be understood that the term “subject” refers to and includes humans and other animals, whether they be alive or once-living. Similarly, the term “body part or other structure” may mean a tissue-containing structure in a living or once-living organism such as a human or other animal.

Likewise, it should be understood that the term “structure” may mean a tissue-containing structure in a living or once-living organism such as a human or other non-human animal.

The term “near” and “in proximity” when referring to placement of coils outside a subject's body may be less than one meter.

It should be understood that the term “magnetizable” and “magnetic” are used interchangeably to indicate a material that can be magnetized.

It should be understood that the term “magnetizable particle” may refer to a particle made of material that exhibits magnetic or electric properties after or during exposure to a magnetic field. It should be understood that the term “particle” means an object smaller than 1 mm, 100 micron, 10 microns, 1 micron, 0.1 microns, or 0.01 microns in the smallest diameter.

As explained above, intra-tumoral injection may assist the body of a subject to mount an immune response to tumor cells that have dispersed elsewhere in the body from the injection site.

However, the ability to deliver intra-tumoral therapy is limited when the tumor foci are deep within a subject's tissue or numerous. Disclosed embodiments provide an effective and efficient mechanism to deliver intra-tumoral therapy to multiple foci.

The term “deep” used herein refers to a distance that varies depending on the ability to administer a drug or other therapeutic payload via injection. For the purposes of this disclosure, the term “deep” is understood to mean that a target in a subject's body is not readily accessible from the surface of the body. An example of such inaccessibility is when the target is not palpable by an observer, or when it is not visible to an observer's naked eye.

In accordance with disclosed embodiments, a therapeutic payload may be included within magnetic carrier particles that may be administered to a subject via, for example, needle injection or intravenously.

In accordance with disclosed embodiments, an apparatus for delivery of therapeutic drug formulations to tumors includes a magnetic system including a Magnetic Resonance Imaging (MRI) system. As described in prior publications and patent applications by A Nacev and I Weinberg, the MRI system is capable of propelling magnetic materials under imaging guidance. See, for example, U.S. Pub. 20130204120, corresponding to U.S. patent application Ser. No. 13/761,200, by I. Weinberg, and entitled “EQUIPMENT AND METHODOLOGIES FOR MAGNETICALLY-ASSISTED DELIVERY OF THERAPEUTIC AGENTS THROUGH BARRIERS,” and U.S. Pat. Pub. 20170227617, corresponding to U.S. patent application Ser. No. 15/427,426, by I. Weinberg and A. Nacev, and entitled “METHOD AND APPARATUS FOR MANIPULATING ELECTROPERMANENT MAGNETS FOR MAGNETIC RESONANCE IMAGING AND IMAGE GUIDED THERAPY,” incorporated herein by reference.

Thus, in accordance with various embodiments, magnetic gradients and fields may be generated by the systems disclosed herein may be used to propel medical treatment payload in a carrier to a target/tumor within a subject's body. Further, the system may be configured to alternately image and propel the carrier-payload. Thus, the MRI system may include a system to apply magnetic fields under imaging guidance that includes permanent magnets, electromagnets, antennas or electro-permanent magnets. Such electro-permanent magnets may at one or more times create a magnetic field configuration for imaging of a subject's body part and then at another set of times create a magnetic field configuration for propulsion of particles. It should be understood that the imaging capability may be through magnetic resonance imaging methods.

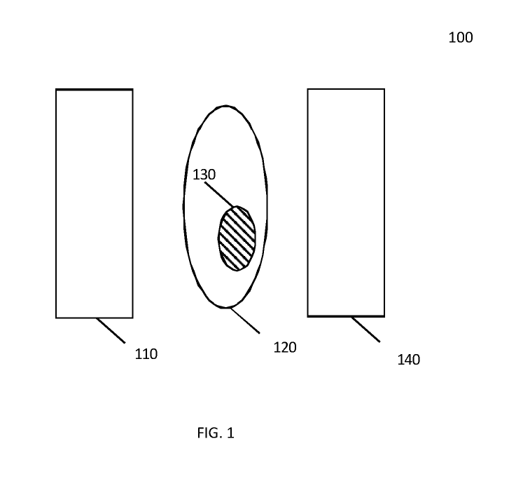

FIG. 1 illustrates a magnetic system 100 configured in accordance with the disclosed embodiments. As shown in FIG. 1, a subject's body part 20, which includes a tumor and/or other target 30, may be inserted between the two-sided magnetic system 100 for image-guided delivery of a therapeutic agent. The magnetic system 100 may include two magnet assemblies 110 and 140. Thus, the magnetic system 100 is represented in FIG. 1 by assemblies 10 and 40. The magnetic systems 100, 200, 300 include these assemblies that represent a two-sided configuration, in which electro-permanent magnets can establish a “static” magnetic field for a short period of time (for example, 1 second) in which protons or other magnetic materials align. Additionally, other coils (for example, gradient or shim or radiofrequency send or receive coils) may be part of the assemblies, and may be used to collect an image of the body part 120, 220, and 320, wherein the body part is in a subject such as a human or animal.

It should be understood that the disclosed apparatus and methodologies may be used in conjunction with other components, for example a computer and/or a power supply and/or coils for generating magnetic and/or electromagnetic fields, in order to attain a desired result of a meaningful image. It is understood that the image may use principles of proton magnetic resonance imaging, or magnetic resonance imaging of other particles (for example, electrons or sodium atoms) or other imaging principles (for example, magnetic particle imaging, or impedance imaging). It is understood that the apparatus may be used to deliver therapy by manipulating magnetizable materials with the magnetic field produced by the device. It should be understood that this manipulation may be performed at one time, and that imaging may be performed at another time, in order to guide the manipulation described above.

It is understood that body part 120 may be the entire body, or may be several body parts treated sequentially. Further, it is understood that the action of the assemblies is under the control of a computer, and may be autonomously targeted to one or more targets on the basis of the magnetic resonance images of the body part and targets. The body part may contain a target 130, 230 and 330 that may be a cancerous tumor, included in such a tumor or otherwise is close proximity to such a tumor, for example, one or more lymph nodes located in close proximity to a lymph node including cancer.

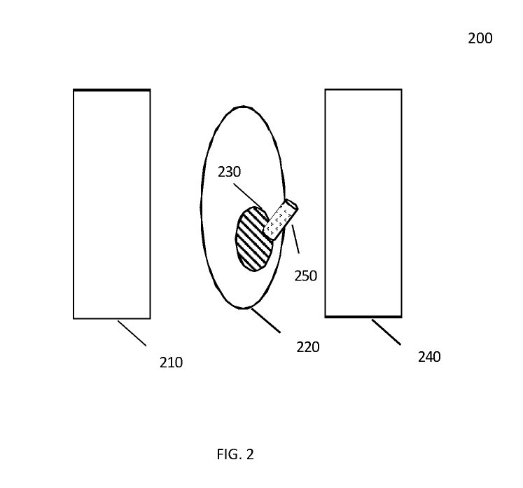

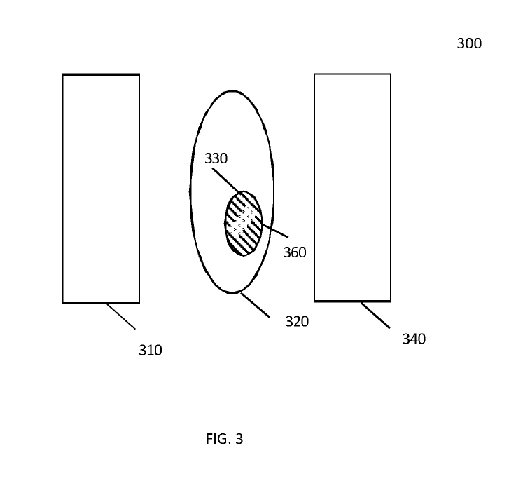

Likewise, as illustrated in FIG. 2, assemblies 210 and 240 similarly correspond to and represent the two-sided magnetic system 200 in accordance with the disclosed embodiments. Further, as illustrated in FIG. 3 assemblies 310 and 340 similarly correspond to and represent the two-sided magnetic system 300 in accordance with the disclosed embodiments. It should be understood that systems 100, 200 and 300 may all have the same features or have different features without diverting from the concepts disclosed herein. Likewise, the assemblies 110, 210, 310 and 140, 240, 340 may all have the same features or different features without diverting from the concepts disclosed herein.

FIGS. 2 and 3 demonstrate method operations of the disclosed embodiments. In FIG. 2, a carrier 250 with magnetic components and a payload is delivered with the aid of magnetic assemblies 210 and 240 to target 230 in the subject's body part 220. FIG. 2 illustrates a magnetic carrier 250 being delivered by operation of the magnetic assemblies 210 and 240, wherein the magnetic fields generated by the assemblies are controlled to propel the magnetic carrier 250 in the subject's body part 220.

It is understood that assemblies 210 and 240 may also provide magnetic particle images of the magnetic carrier 250, in which “magnetic particle imaging” may be the conventionally known and classical method of detecting signals from magnetization of the particles (see U.S. Pat. No. 7,482,807, by B. Gleich et al. and entitled “METHOD OF DETERMINING A SPATIAL DISTRIBUTION OF MAGNETIC PARTICLES;”), or may be a method of rapid magnetic resonance imaging as described in U.S. Pub. 20170139024, corresponding to U.S. patent application Ser. No. 15/352,164, A. Nacev and entitled “METHOD AND APPARATUS FOR HIGH SLEW RATE SINGLE POINT MAGNETIC RESONANCE IMAGING OF MAGNETIZABLE NANOPARTICLES” (incorporated herein by reference in its entirety).

The magnetic fields for propulsion applied by assemblies 210 and 240 may be generated and controlled as described in U.S. Pat. No. 9,380,959 “MRI-GUIDED NANOPARTICLE CANCER THERAPY APPARATUS AND METHODOLOGY” (incorporated herein by reference). The magnetic carrier 250 may contain a payload, for example an oncolytic virus. The magnetic carrier may have multiple magnetic features that enable effective penetration of tissue through rotation, as described in U.S. Pub. 20170069416 corresponding to U.S. patent application Ser. No. 14/930,126, filed by L. Mair, A. Nacev and I. Weinberg, and entitled “Method and apparatus for non-contact axial particle rotation and decoupled particle propulsion”. The magnetic carrier 250 may be introduced non-invasively, for example through a natural orifice like the nose.

FIG. 3 shows method operations performed in accordance with a disclosed embodiment, wherein one or more portions of the carrier 250 (shown in FIG. 2) dissolves and the payload 360 is delivered to the target 330. Thus, as shown in FIG. 3, the walls of the carrier 250 (shown in FIG. 2) have dissolved, and the payload 360 is delivered to the target 330 in the subject's body part 320.

As explained above with reference to FIG. 2, the magnetic assemblies 310 and 340 are configured to image the target/tumor 330.

One or more portions of the carrier 250 (shown in FIG. 2) or a wall of the carrier may dissolve because the portion is made of iron, which dissolves at a rate of about one micron per hour in oxygenated fluids. Alternatively, the portion of the carrier may be made of a plastic that dissolves in the body at a known rate under certain conditions. For example, the dissolving portion may be manufactured of a polymer that dissolves in several minutes when the carrier 250 is heated by magnetic pulses or currents generated by assemblies 310 and 340.

Accordingly, as a result of the targeted delivery of the carrier and dissolving of a portion of the carrier/carrier wall enables delivery of a medical treatment payload to a target/tumor location with a body part of a subject. Thus, the disclosed embodiments may be utilized to accomplish intra-tumoral injection of one or more target sites with a medical treatment payload, and may, therefore, be more effective than systemic administration of that payload.

It should be understood that, optionally, one or more magnetic fields applied by the magnetic system disclosed herein to a body part of a subject may be so rapidly applied so as not to cause unpleasant nerve stimulation, as taught by I. Weinberg in issued U.S. Pat. No. 8,154,286, entitled “APPARATUS AND METHOD FOR DECREASING BIO-EFFECTS OF MAGNETIC FIELDS” and related applications related through priority rights by I. Weinberg, incorporated herein by reference.

It should be understood that the operations explained herein may be implemented in conjunction with, or under the control of, one or more general purpose computers running software algorithms to provide the presently disclosed functionality and turning those computers into specific purpose computers.

Moreover, those skilled in the art will recognize, upon consideration of the above teachings, that the above exemplary embodiments may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out the above-described method operations and resulting functionality. In this case, the term non-transitory is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While certain illustrative embodiments have been described, it is evident that many alternatives, modifications, permutations and variations will become apparent to those skilled in the art in light of the foregoing description. Accordingly, the various embodiments of, as set forth above, are intended to be illustrative, not limiting. Various changes may be made without departing from the spirit and scope of the invention.

What is claimed:

1. A method for delivering a payload to one or more targets in one or more body parts of a subject under magnetic imaging guidance, the method comprising:
administering one or more magnetic carrier particles including one or more payloads to the subject;
performing image-guided magnetic delivery of the one or more of the plurality of magnetic carrier particles to the one or more targets using at least one image-guidance component by propelling the plurality of magnetic carrier particles at least in part by applying a magnetic field to the subject's body part using one or more coils,
wherein one or more portions of the magnetic carrier particles dissolve after positioning of the plurality of magnetic carrier particles at the one or more targets in the one or more body parts.

2. The method of claim 1, wherein the one or more targets are tumor foci.

3. The method of claim 1, wherein the one or more targets are foci of metastatic cancer.

4. The method of claim 1, wherein the plurality of magnetic carrier particles are introduced into the one or more body parts with minimal invasiveness.

5. The method of claim 1, wherein the one or more body parts are deep in the subject's body.

6. The method of claim 1, wherein the plurality of magnetic carrier particles are guided autonomously by a computer based on magnetic images collected by the at least one image-guidance component.

7. The method of claim 1, wherein the image-guided magnetic delivery of the one or more of the plurality of magnetic carrier particles to the one or more targets injects the payload into one or more tumor foci located at the one or more targets thereby facilitating the subject's body mounting an immune response to cells similar to cells in the one or more tumor foci.

8. The method of claim 1, wherein the plurality of magnetic carrier particles are administered by injection or intravenously.

9. The method of claim 1, wherein the at least one image-guidance component comprises an MRI system that includes the at least one magnetic coil.

10. The method of claim 1, wherein each of the plurality of magnetic carrier particles includes a portion that dissolves in the body part to release the payload.

11. The method of claim 1, where the payload contains an oncolytic virus.

12. The method of claim 1, where the plurality of magnetic carrier particles contains features for decoupled rotation and propulsion.

13. An apparatus for delivering a payload to one or more targets in a subject's body part under magnetic imaging guidance, the apparatus comprising:
one or more coils disposed near to the subject's body part and outside the subject's body part;
at least one image-guidance component positioned in proximity to the subject's body part; and
a controller coupled to the one or more coils and configured to control the one or more coils to generate a magnetic field,
wherein the at least one image-guidance component provides imaging data that enables image-guided magnetic delivery of the one or more of the plurality of magnetic carrier particles administered into the subject's body part to the one or more targets using the at least one image-guidance component by propelling the plurality of magnetic carrier particles at least in part by applying the magnetic field to the subject's body part using the one or more coils,
wherein one or more portions of the magnetic carrier particles dissolve after positioning of the plurality of magnetic carrier particles at the one or more targets in the one or more body parts.

14. The apparatus of claim 13, wherein the one or more targets are tumor foci.

15. The apparatus of claim 13, wherein the one or more targets are foci of metastatic cancer.

16. The apparatus of claim 13, wherein the plurality of magnetic carrier particles are introduced into the one or more body parts with minimal invasiveness.

17. The apparatus of claim 13, wherein the one or more body parts are deep in the subject's body.

18. The apparatus of claim 13, wherein the plurality of magnetic carrier particles are guided autonomously by a computer based on magnetic images collected by the at least one image-guidance component.

19. The apparatus of claim 13, wherein the image-guided magnetic delivery of the one or more of the plurality of magnetic carrier particles to the one or more targets injects the payload into one or more tumor foci located at the one or more targets thereby facilitating the subject's body mounting an immune response to cells similar to cells in the one or more tumor foci.

20. The apparatus of claim 13, wherein the plurality of magnetic carrier particles are administered by injection or intravenously.

21. The apparatus of claim 13, wherein the at least one image-guidance component comprises an MRI system that includes the at least one magnetic coil.

22. The apparatus of claim 13, wherein each of the plurality of magnetic carrier particles includes a portion that dissolves in the body part to release the payload.

23. The apparatus of claim 13, where the payload contains an oncolytic virus.

24. The apparatus of claim 13, where the plurality of magnetic carrier particles contains features for decoupled rotation and propulsion.
